(19) United States
(12) Patent Application Publication
(10) Pub. No.: US 2002/0193738 A1
(43) Pub. Date: Dec. 19, 2002

(54) Heat exchange catheter and the employment thereof

(75) Inventors: Vaso Adzich, Yorba Linda, CA (US); Nora Pham, Lake Forest, CA (US)

Correspondence Address: LYON & LYON LLP, 633 WEST FIFTH STREET, SUITE 4700, LOS ANGELES, CA, 90071, US

(73) Assignee: ALSIUS CORPORATION

(21) Appl. No.: 09/883,869

(22) Filed: Jun. 18, 2001

(51) Int. Cl. A61F 7/12

(52) U.S. Cl. 604/113

(57) ABSTRACT

A catheter adapted for heat exchange within a body includes a shaft with at least one input lumen and at least one output lumen in fluid communication with one or more elongate elements which each include a heat transfer lumen. The elongate element is arranged in one or more lengths which include heat transfer lumens being folded into first elongate substantially parallel and juxtaposed portions arranged either in series or parallel or both with adjacent wings. The juxtaposed portions are arranged together in planes extending radially from the center axis of the assembly and are equiangularly placed about that axis. The ends of the portions may be bonded together to create the radial plane. An infusion lumen may extend through the heat transfer assembly with an open end for infusing into the blood stream or sampling therefrom. The efficiency of the heat transfer region is maximized through the elongate thin-walled elements in that surface area is increased and turbulence may be maintained with sufficient input pressure. The employment of the catheter includes its preparation, insertion and expansion with fluid pressure. Additionally, the system may be pressurized to insure turbulent flow through the heat transfer lumens. Use in cardiac surgery and fever control is contemplated with placement of the catheter in the groin, the chest or neck of the patient.

### BACKGROUND OF THE INVENTION

The field of the present invention is heat transfer catheters and the methods of their use.

The advantageous use of hypothermia in medical procedures is known. For example, a reduction in metabolic rate of a body may be achieved through the lowering of body temperature. Reduced metabolism makes it possible to more easily accommodate lengthy operative procedures. In stroke, trauma and several other pathological conditions, hypothermia also reduces the permeability of the blood/brain barrier. It inhibits release of damaging neurotransmitters and also inhibits calcium-mediated effects. Further, hypothermia inhibits brain edema and lowers intracranial pressure. Success has been observed for patients suffering from severe brain trauma or from ischemia caused by stroke or heart attack when the patient is cooled below normal body temperature (38° C.).

Hypothermic treatment has been typically addressed systemically, meaning that the overall temperature of the entire body is lowered. An example of this systemic approach includes catheters for transferring heat to or from body fluid in a body conduit such as blood flowing within a patient's vessel, as disclosed by Ginsburg in U.S. Pat. No. 5,486,208. A closed-loop heat exchange catheter is also disclosed by Saab in U.S. Pat. No. 5,624,392. A cooling device used systemically in the circulatory system is known to be more efficient since the entire volume of the body is constantly perfused with the cold fluid at a capillary level.

Various other means of cooling the body have been employed. Such means include cooling blankets, ice water, bladder lavages, ice baths, esophageal catheters and associated methods. However, these less systemic devices require a considerable time to cool the body since the primary heat transfer occurs through the skin or the skull.

In other medical situations, it may be desirable to raise the patient's body temperature. For example, a patient may suffer from unintended hypothermia and may need to be warmed to a normothermic temperature. These results can be obtained in innervascular heating through a heat exchange catheter.

Certain limiting factors affect the capability of heat exchange catheters to provide maximum cooling or heating of a patient safely and in a controlled manner. The rate of heat transfer depends on such factors as the volumetric flow rates of the body fluid and the heat exchange fluid, and the temperature difference between the heat exchanger and the body fluid. Other factors include the convection heat transfer coefficient of the two fluids involved in the heat exchange, the thermal conductivity and thickness of the barrier between the two fluids and the residence time of the heat transfer. Increasing the cooling or heating rate may be accomplished by, for example, increasing the size of the heat transfer surface or by increasing the temperature difference between the heat exchange fluid and the body fluid. Increasing the size of the heat transfer surface, however, may result in blocking the flow of blood in a blood vessel. Such blockage could cause, inter alia, thrombosis. Increasing the size of the heat transfer surface is further complicated by the widely varying sizes of blood vessels among different patients. Further, excessively increasing the temperature difference between the heat exchange fluid and blood may lead to undesirable results, such as thermal damage to the blood vessel wall. Blood protein degeneration and coagulation may also occur. The rate of heat transfer is also affected by the fluid flow and distribution within the heat exchanger. Turbulent flow provides greater heat transfer through the wall of the device as does any fluid mixing and full flow. Lack of mixing in either the heat transfer liquid or the body fluid reduces efficiency.

### SUMMARY OF THE INVENTION

The present invention is directed to a heat exchange catheter and the method of its employment. A catheter to exchange heat with a body fluid employs input and output lumens and at least one elongate element including a heat transfer lumen for heat transfer with the body fluid.

In a first separate aspect of the present invention, the elongate element is folded into elongate substantially parallel and juxtaposed portions. The arrangement provides increased surface area and flow control. There may be a plurality of such elongate elements. Further, the portions can be arranged in planes extending radially from the centerline of the catheter, equiangularly spaced about the catheter for centering within a vessel or other body conduct and/or arranged in a helix. The arrangement provides for the capability of turbulent flow, other fluid mixing and full flow throughout the heat transfer lumen.

In a second separate aspect of the present invention, each elongate element may include two lengths forming elongate substantially parallel and juxtaposed portions. In this way, series flow through multiple heat transfer zones may be accomplished. Multiple input lumens may also respectively couple with elongate elements to form parallel flow. A combination of these arrangements is also contemplated with both series and parallel flow.

In a third separate aspect of the present invention, at least one elongate element is folded into first elongate substantially parallel and juxtaposed portions and includes an infusion lumen with an opening at one end for both efficient heat transfer and infusion or fluid sampling.

In a fourth separate aspect of the present invention, the heat exchange catheter includes a shaft with the at least one elongate heat transfer element including elongate substantially parallel and juxtaposed portions extending longitudinally of the shaft. The juxtaposed portions may lie in a plane extending radially from the shaft. Multiple such elements may be equiangularly spaced about the shaft and possibly formed into a helix.

In a fifth separate aspect of the present invention, the heat exchange catheter is employed by insertion into a body conduit, the directing of liquid through the catheter and through an elongate element folded into first elongate substantially parallel and juxtaposed portions and the raising of pressure to expand fully the elongate element. The liquid pressure may be raised in the elongate conduits to achieve turbulent flow and/or any fluid mixing and full flow. The employment of the catheter may also include insertion with a sheath or the use of a wire guide in placing the catheter in the neck, chest or groin of the body.

In a sixth separate aspect of the present invention, combinations of any of the foregoing aspects and features are contemplated.

Accordingly, it is an object of the present invention to provide improved heat transfer catheters and methods of their employment. Other and further objects and advantages will appear hereinafter.

### BRIEF DESCRIPTION OF THE DRAWINGS

FIG. 1 is a side view of a catheter for heat exchange within the body.

FIG. 2 is a cross-sectional view of a catheter.

FIG. 3 is a cross-sectional view of a catheter.

FIG. 4 is a cross-sectional view of a catheter.

FIG. 5 is a schematic perspective view of a catheter with heat transfer portions in a first arrangement.

FIG. 6 is a schematic perspective view of a catheter with heat transfer portions in a second arrangement.

FIG. 7 is a schematic perspective view of a catheter with heat transfer portions in a third arrangement.

FIG. 8 is a cross-sectional side schematic of one arrangement of a catheter with heat transfer elements.

FIG. 9 is a cross-sectional side schematic of one arrangement of a catheter with heat transfer elements.

FIG. 10 is a cross-sectional side schematic of one arrangement of a catheter with heat transfer elements.

FIG. 11 is a cross-sectional side schematic of one arrangement of a catheter with heat transfer elements.

FIG. 12 is a cross-sectional side schematic of one arrangement of a catheter with heat transfer elements.

FIG. 13 is a cross-sectional side schematic of one arrangement of a catheter with heat transfer elements.

FIG. 14 is a cross-sectional side schematic of one arrangement of a catheter with heat transfer elements.

FIG. 15 is a side schematic of a catheter with a heat transfer element.

FIG. 16 is a schematic side view of a cathether and heat transfer element configuration.

FIG. 17 is a schematic side view of a cathether and heat transfer element configuration.

FIG. 18 is a schematic side view of a cathether and heat transfer element configuration.

FIG. 19 is a representative graph of flow rate versus Reynolds number.

FIG. 20 is a schematic perspective view of a catheter with heat transfer portions in a first arrangement defining a helix.

FIG. 21 is a side elevation view of a patient lying in a prone position with a heat exchange catheter in place in the groin. Placements in the chest and neck are shown in phantom.

FIG. 22 is a schematic illustration of a catheter with a sheath.

FIG. 23 is a schematic illustration of a catheter associated with a wire guide.

### DETAILED DESCRIPTION OF THE PREFERRED EMBODIMENTS

Turning in detail to the drawings, a plurality of cathether arrangements are illustrated. In all such arrangements, the catheters include hollow elongate heat transfer elements with heat transfer lumens being folded into first elongates substantially parallel and juxtaposed portions.

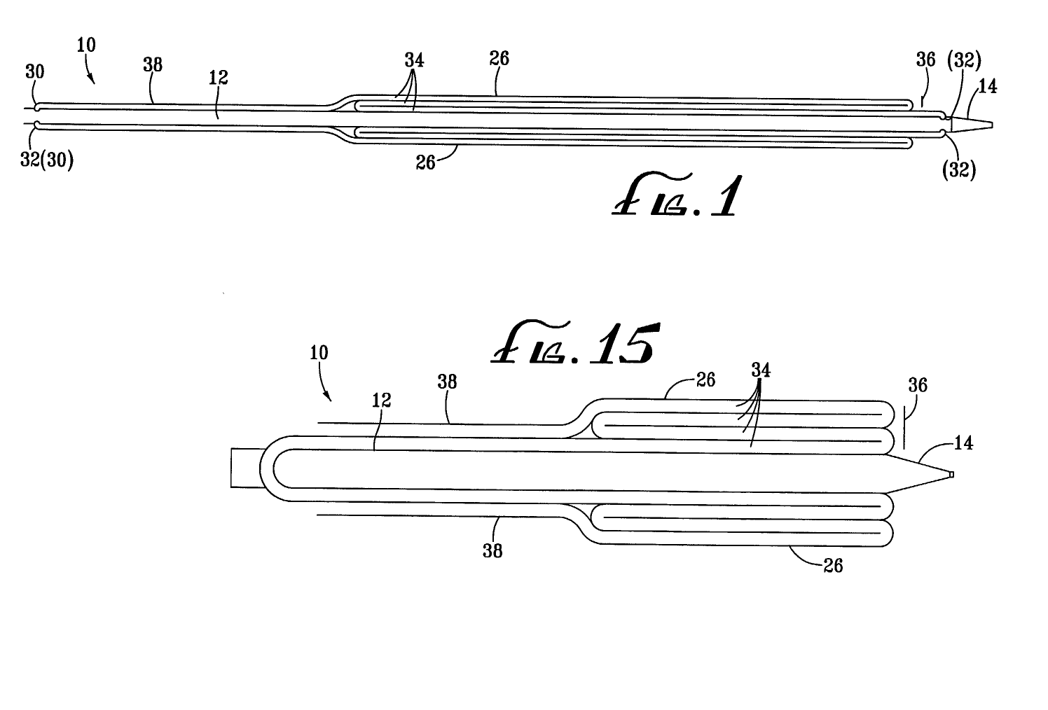

FIG. 1 illustrates a catheter, generally designated 10, including a shaft 12. The shaft 12 is flexible and yet allows entry into body conduits in a conventional fashion. There is a blunt head 14 at the distal end of the shaft 12 for ease of insertion. A hub or other fluid attachment means (not shown), preferably with standard fittings for coupling with sources of pressurized heated or cooled fluid and returns, is arranged at the proximal end of the catheter 10.

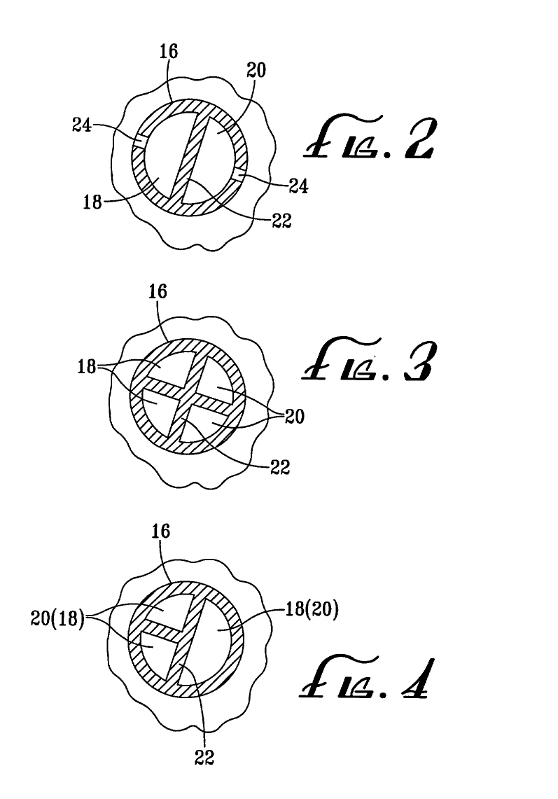

Three possible cross-sections of the shaft 12 are illustrated in FIGS. 2, 3 and 4. Each figure illustrates a shaft outer wall 16, an input lumen 18 and an output lumen 20. The lumens 18 and 20 extend longitudinally along the shaft within the outer wall 16. In FIG. 2, there is a single input lumen 18 and a single output lumen 20. In FIG. 3, there are two input lumens 18 and two output lumens 20. In FIG. 4, there is either one input lumen 18 and two output lumens 20 or two input lumens 18 and one output lumen 20. The arrangements of lumens within the outer wall 16 are defined by septums 22. Obviously, the requirements of flow may suggest variations on the subtended angles of each lumen within the circular wall 16. Additionally, other septum configurations are possible. The shaft 12 includes ports 24 through the outer wall 16 which may provide supply or return depending on the manifolding of the catheter.

Hollow elongate heat transfer elements 26 are arranged about the shaft 12 as shown in FIG. 1. Heat transfer lumens, preferably of about 6 to 14 French, extend through the hollow elongate elements 26. They each include a first end 30 and a second end 32. The ends 30 and 32 are shown to extend to ports 24 through the shaft wall 16.

Each element 26 includes a first length which is folded between the first and second ends to create elongate substantially parallel and juxtaposed portions 34. These portions 34 are shown to lie in a plane extending radially from the shaft 12 to form, in the embodiment of FIG. 1, two wings 36. The elements 26 are illustrated in this embodiment to be substantially uniform in cross section and of substantially the same size as the ports 24.

The elements 26 are of thin wall material, typically employed in the construction of balloons such as illustrated in U.S. Pat. No. 6,126,684, the disclosure of which is incorporated herein by reference. The material is conveniently polyethylene or polyurethane. The catheters are manufactured with the elements completely collapsed on the side wall of the shaft 12. The imposition of pressure into the catheter then expands these elements 26 once the catheter has been properly positioned within the body. Vacuum may also be applied to achieve relatively complete collapse after use for purposes of extraction. The individual portions making up the folded length of the elements 26 may be bonded together along mutual tangents with adjacent portions 34 or the shaft wall 16, for the innermost such portion 34. To increase efficiency, it may be advantageous to simply glue the ends of each of the portions 34 together and leave the central length of each of the portions 34 unattached to the adjacent portion or structure.

Thus, heat transfer fluid is able to flow first through the shaft 12 and then through the ports 24 in the wall 16 so as to enter the first end 30 of each of the elements 26. Heat transfer fluid then flows through the heat transfer lumens in the portions 34, three being shown in each element in FIG. 1, and exits through the second end 32 into a port 24 through the wall 16 of the shaft 12. The first ends 30 of the elements 26 may be in communication with the same input lumen 18 as satisfied by the arrangements of FIG. 2 and FIG. 4. Similarly, the second ends 32 of the elements 26 may be in communication with the same output lumen 20, also satisfied by the configurations of FIG. 2 and FIG. 4. In these configurations, parallel flow from common input and output lumens is achieved in at least one of the input and the output. Alternatively, each end 30 and 32 of the two elements 26 may be in communication with a separate input lumen 18 and output lumen 20 as satisfied by the arrangement illustrated in FIG. 3. In this case, the flow is parallel from separate sources and to separate returns. The embodiment shown in FIG. 1 can be arranged in several ways. As described above, the ends 30 and 32 are shown to be at the proximal end of the heat transfer zone. As shown parenthetically in the same figure, the ports 24 may be arranged such that input (or output) may be at the proximal end of the heat transfer zone and the output (or input) may be at the distal end of the heat transfer zone.

Because of the size of body conduits and the arrangement of the plurality of portions 34 forming each wing 36, the heat transfer lumens defined within the elements 26 are relatively small, far smaller in cross-sectional flow area than balloons that have been contemplated in the past. As such, flow may be induced at sufficient pressure to achieve turbulent flow. Turbulent flow results in a full mixing of the heat transfer fluid within the lumens and without quiescent areas. The pressure necessary for inducing turbulence in the heat transfer elements 26 and 28 may be accomplished through a combination of empirical observations and calculations. Assuming specific pressures can be empirically correlated with flow rates, critical Reynolds numbers can be correlated with critical flow speeds necessary for turbulence. A representative graph of flow rates vs. Reynolds numbers is illustrated in FIG. 19. The uniformity in the cross section of each heat transfer lumen and the comparable size of the ports 24 further insure that there are no quiescent areas within the elements 26 and movement and mixing also occur under laminar conditions.

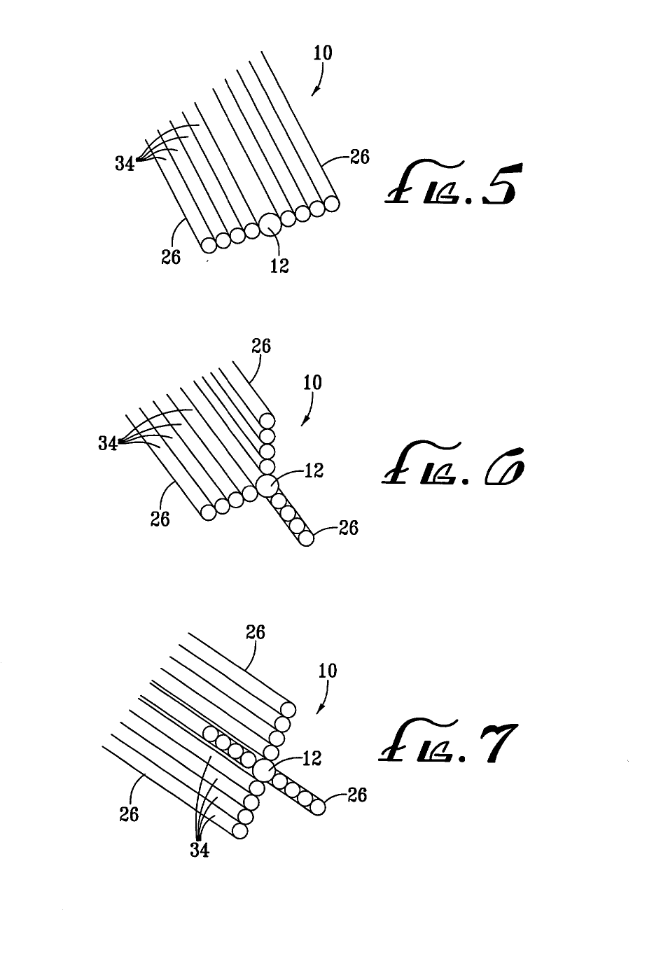

A number of variations on the structure illustrated in FIG. 1 can be employed. FIGS. 5, 6 and 7 show arrangements, truncated for simplicity, where a shaft 12 includes equiangularly spaced wings. Four elongate substantially parallel and juxtaposed portions 34 are shown in each wing 36. However, other pluralities may be appropriate as well. In each of the three figures, the wings are arranged in planes equiangularly spaced about the shaft 12 and extend radially therefrom.

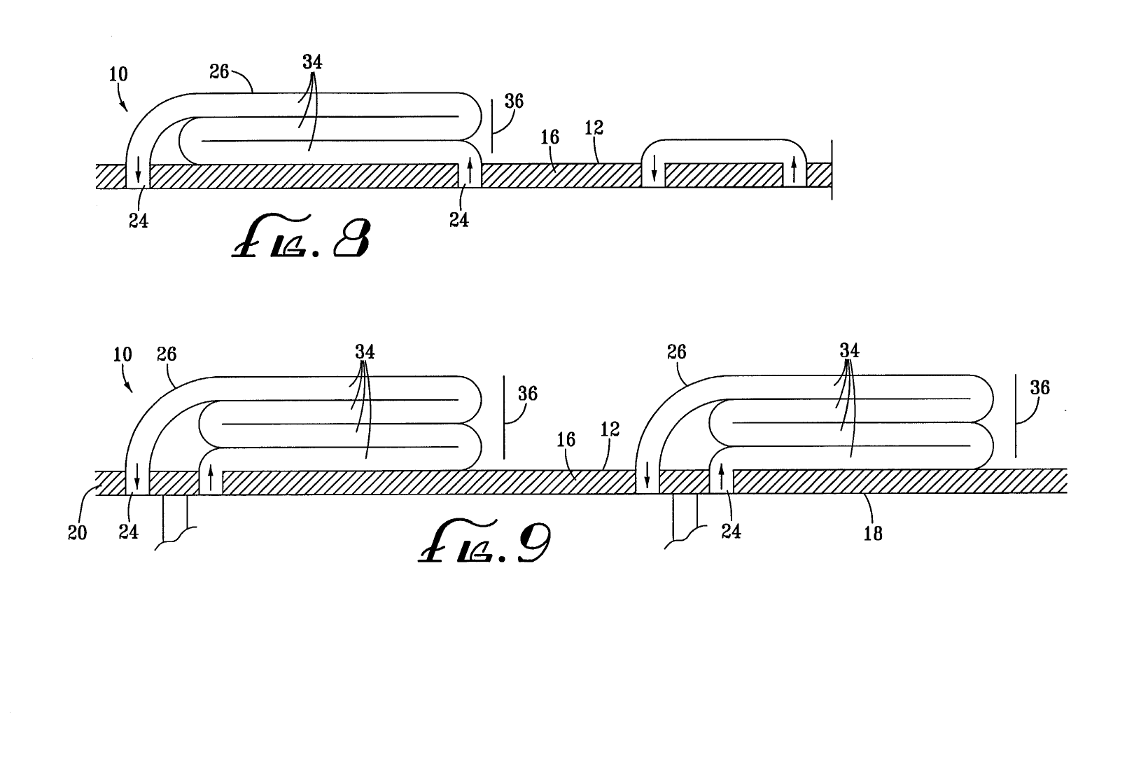

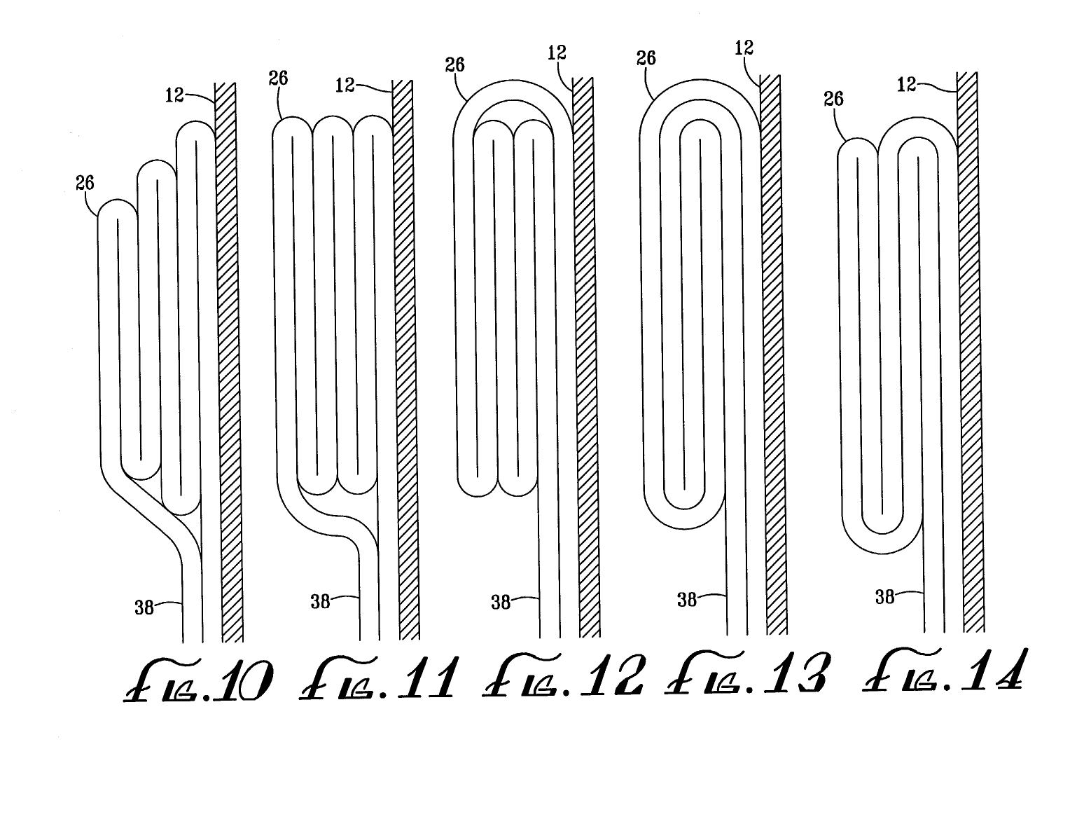

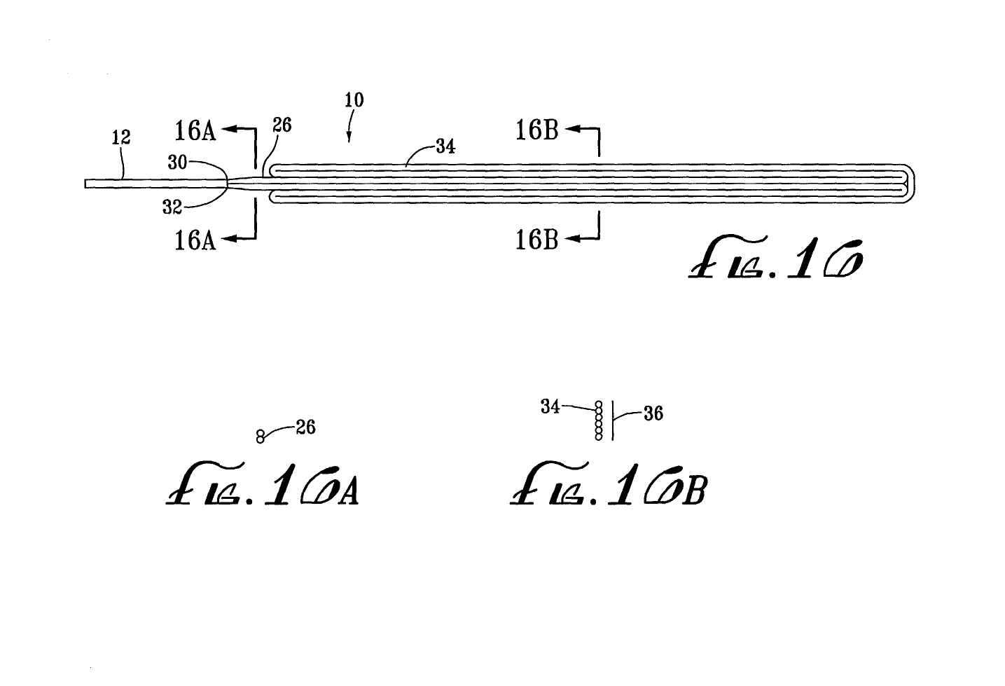

The wings 36 may be created by folded lengths of the hollow elongate heat transfer elements 26. In FIG. 8, a fold arrangement similar to that of FIG. 1 is illustrated. Another length 38 is shown without folding. Several other fold patterns are illustrated in FIGS. 10 through 14. In FIG. 9, two four-portion wings 36 are illustrated which may be arranged in series. A block in the input lumen 18 between the first end 30 and the second end 32 on each of the wings 36 would achieve the series flow arrangement. FIG. 15 illustrates a series flow arrangement where the wings 36 are not displaced longitudinally along the catheter from one another. Rather, the wings 36 are angularly displaced and laterally aligned.

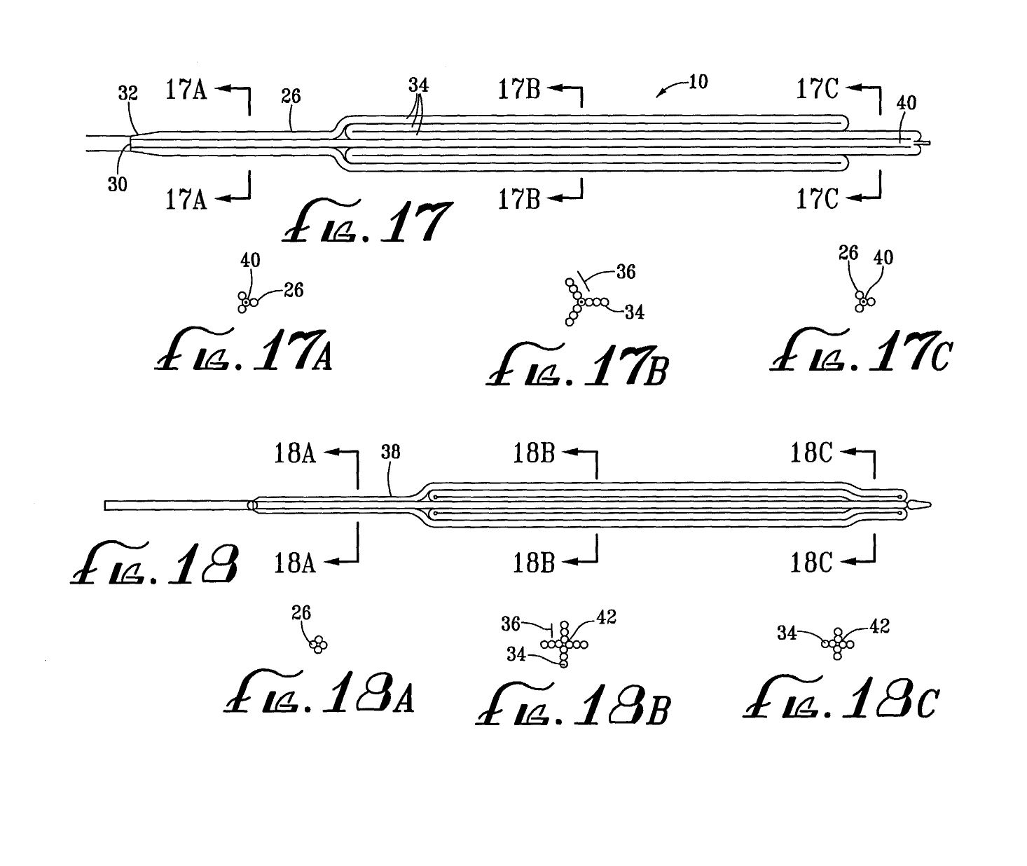

Full assemblies are illustrated in FIGS. 16 through 18. FIGS. 16A and 16B illustrate the cross-sectional layout at the indicated respective locations of the catheter 10 illustrated in FIG. 16. The shaft 12 includes an input lumen and an output lumen. A hollow elongate heat transfer element 26 extends from the input lumen 18 and the output lumen 20. A first end 30 of the hollow elongate heat transfer element 26 is in fluid communication with the input lumen 18 while a second end 32 of the hollow elongate heat transfer element 26 is in fluid communication with the output lumen 20. The hollow elongate heat transfer element 26 is folded as shown in the figure to create elongate substantially parallel and juxtaposed portions 34 lying within a plane and fixed together at least at each end. For insertion, a sheath may be employed thereabout with the portions 34 in fully collapsed condition. A wire guide may be used with or without the sheath for insertion.

FIG. 17 discloses a three-wing arrangement with the shaft 12 providing an input lumen and an output lumen. The three wings 36 defined in this arrangement share a common first end 30 including a first elongate substantially parallel portion 40 extending the length of the heat transfer section. Adjacent elongate substantially parallel and juxtaposed portions 34 then divide from this portion 40. The portion 40 may be appropriately sized to accommodate the flow. Three such folded portions 34 make up the remainder of the hollow elongate heat transfer element 26 along with three output portions extending from the folded portions to the shaft 12.

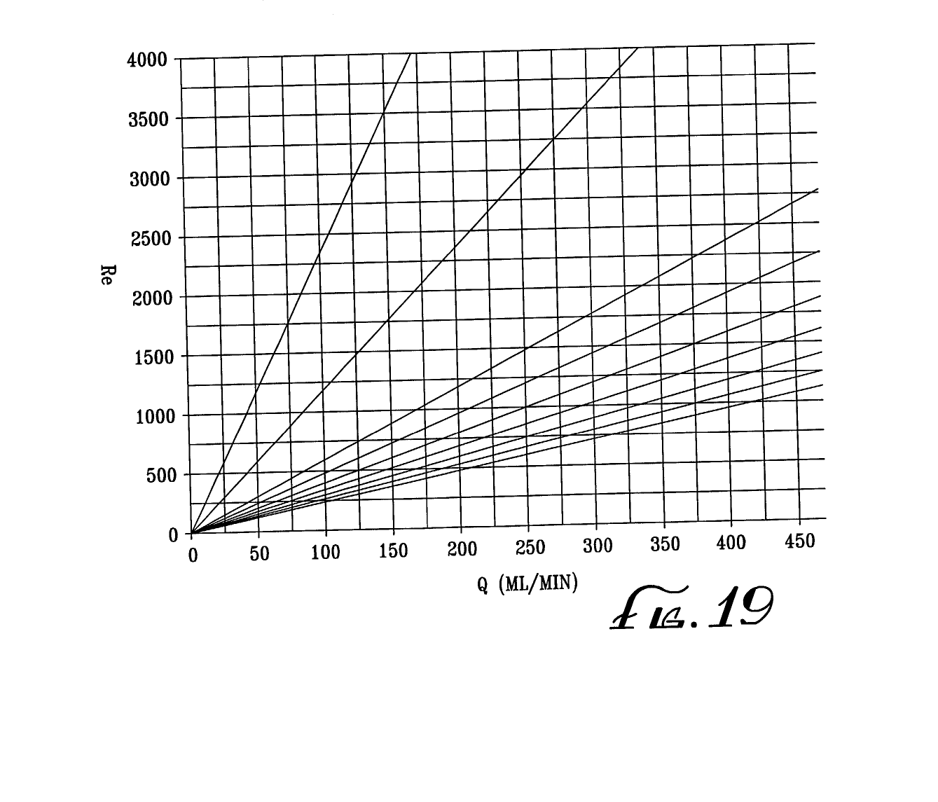

FIG. 18 illustrates the heat transfer end of a catheter 10 having four wings 36 equiangularly placed about a common central body 42. The central body 42 may include an infusion lumen having an opening at the distal end thereof. The central body may be a wire guide employed only for positioning or an infusion lumen capable of including a wire guide to provide dual functions. The infusion lumen of the central body 42 extends longitudinally of the central body 42 within an outer wall. The shaft 12 further includes either one or two input lumens and one or two output lumens in addition to a central infusion lumen. In either instance, there are two hollow elongate elements with each of the two hollow elongate heat transfer elements 26 defining two wings 36. A first length of each element is folded to define three portions lying in a plane extending radially from the center axis of the assembly. This first portion extends in fluid communication from an unfolded length 38 in communication with each input lumen at a first end 30. The first lengths of each of the elements 26 terminate near the distal end of the assembly, crossing over to a second length of the same element 26 defining a second wing 36. The second length is also folded into three portions 34 also defining wings 36. These portions terminate in two unfolded lengths 38 extending to the shaft 12. With the central body 42 providing an infusion lumen, the assembly is able to provide heat transfer, infusion and body fluid sampling with a single placement.

FIG. 20 is unremarkable over the prior embodiments with the exception that the wings 36 are arranged in a helix shape while extending longitudinally of the shaft. The helix shape is understood to cause certain mixing of blood flow past the catheter.

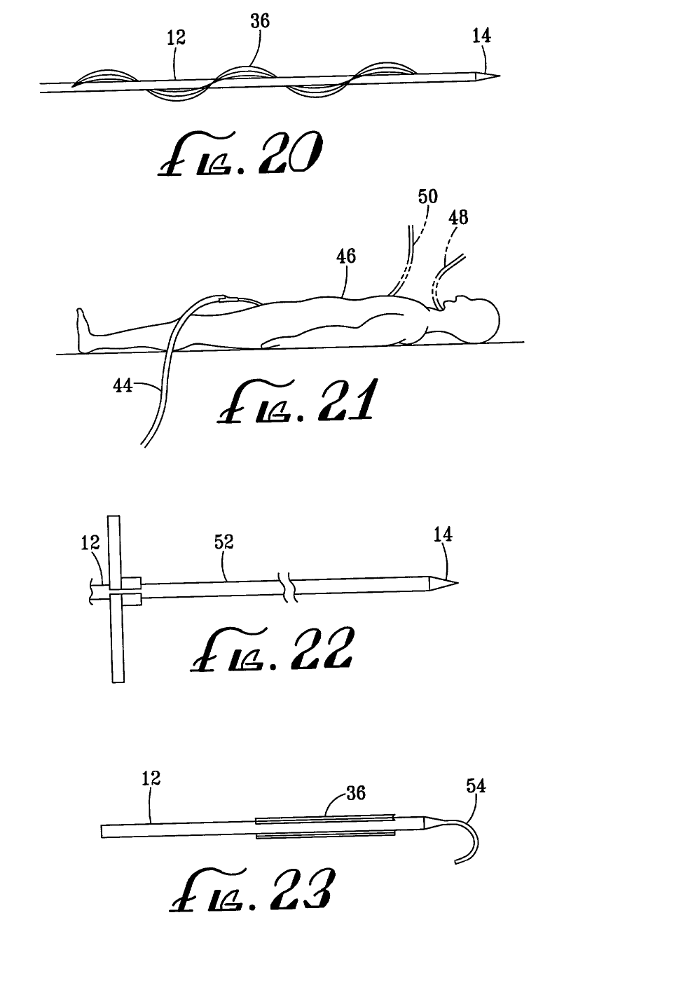

With the foregoing catheters, advantageous employment thereof for heat transfer within the body can be accomplished. The catheter is prepared and inserted into a body conduit. This insertion may be into the groin 44 of the body 46 as illustrated in full in FIG. 21. Shown in phantom are alternate locations for insertion in the neck 48 and the chest 50. Flow is then directed in seriatim through the input lumen 18, a first port 24, the hollow elongate heat transfer element 26, a second port 24 and the output lumen 20. This direction of liquid is accompanied by raising the pressure of the heat transfer fluid to expand fully the thin-walled portions 34. Sufficient pressure may be supplied to achieve turbulent flow within the heat transfer lumen in each element 26. The system may be used for cooling the body coincident with cardiac surgery or for fever control. Conventional placements may be made of the catheter within the body by either removing a sheath 52 after the catheter is inserted (FIG. 22) or by withdrawing a wire guide 54 after inserting the catheter (FIG. 23).

Thus, improved catheters and methods for their use have been disclosed. While embodiments and applications of this invention have been shown and described, it would be apparent to those skilled in the art that many more modifications are possible without departing from the inventive concepts herein. The invention, therefore is not to be restricted except in the spirit of the appended claims.

What is claimed is:

1. A catheter adapted to exchange heat with a body fluid flowing through a body conduit, the catheter comprising
at least one input lumen;
at least one output lumen;
at least one elongate element including a heat transfer lumen extending longitudinally therethrough with first and second ends, each heat transfer lumen being in fluid communication with one of the at least one input lumen at the first end and in fluid communication with one of the at least one output lumen at the second end, each of the at least one elongate element further including a first length between the first and second ends being folded into first elongate substantially parallel and juxtaposed portions.

2. The catheter of claim 1, there being a plurality of the elongate elements.

3. The catheter of claim 2, there being two elongate elements.

4. The catheter of claim 2, there being three elongate elements.

5. The catheter of claim 2, there being four elongate elements.

6. The catheter of claim 2, there being a plurality of the at least one input lumen, each heat transfer lumen being in fluid communication with one of the input lumens at the first end, respectively.

7. The catheter of claim 2, there being a plurality of the at least one output lumen, each heat transfer lumen being in fluid communication with one of the output lumens at the second end, respectively.

8. The catheter of claim 2, the elongate substantially parallel and juxtaposed portions of the elongate elements defining planes, respectively.

9. The catheter of claim 8, the planes defined by the elongate portions of the elongate elements, respectively, being equiangularly spaced about a common axis.

10. The catheter of claim 8, the elongate elements each being formed as a helix.

11. The catheter of claim 8, the elongate elements each including at least three elongate substantially parallel and juxtaposed portions.

12. The catheter of claim 1, each of the at least one elongate element further including a second length between the first and second ends being folded into second elongate substantially parallel and juxtaposed portions.

13. The catheter of claim 12, there being a plurality of the input lumens, each heat transfer lumen being in fluid communication with one of the input lumens, respectively, and each heat transfer lumen being in fluid communication with one of the output lumens, respectively.

14. The catheter of claim 13, the first and second lengths being angularly displaced and laterally aligned.

15. The catheter of claim 12, the elongate first and second substantially parallel and juxtaposed portions of the elongate elements defining planes, respectively.

16. The catheter of claim 1, each elongate substantially parallel portion being bonded to the adjacent elongate substantially parallel portion at the ends thereof.

17. The catheter of claim 1, each elongate substantially parallel portion being thin walled and collapsible under fluid pressure within the body with the heat transfer lumens at atmospheric pressure.

18. The catheter of claim 1, the at least one elongate element including at least three first elongate substantially parallel and juxtaposed portions.

19. The catheter of claim 1 further comprising
an infusion lumen extending parallel with the input lumen and with the output lumen and including an opening at one end thereof adjacent one of the first and the second ends.

20. The catheter of claim 1, the at least one input lumen including at least one input port and the at least one output lumen including at least one output port, the at least one elongate element being coupled at the first end to one of the at least one input port and coupled at the second end to one of the at least one output port, each heat transfer lumen having a substantially uniform cross section between the input port and the output port.

21. A catheter adapted to exchange heat with a body fluid flowing through a body conduit, the catheter comprising
at least one input lumen;
at least one output lumen;
hollow elongate heat transfer elements, each hollow elongate heat transfer element having first and second ends, being in fluid communication with one of the input lumens at the first end, being in fluid communication with one of the output lumens at the second end and being folded between the first and second ends into elongate substantially parallel and juxtaposed portions lying in a plane.

22. The catheter of claim 21, the hollow elongate elements being equiangularly spaced about a common axis.

23. The catheter of claim 21, the hollow elongate elements each being formed as a helix.

24. The catheter of claim 21, each elongate substantially parallel portion being bonded to the adjacent elongate substantially parallel portion at the ends thereof.

25. The catheter of claim 21, each elongate substantially parallel portion being thin walled and collapsible under fluid pressure within the body with the heat transfer lumens at atmospheric pressure.

26. The catheter of claim 21, there being a plurality of the input lumens and a plurality of the output lumens, each first end being in fluid communication with one of the input lumens, respectively, and each second end being in fluid communication with one of the output lumens, respectively.

27. The catheter of claim 21, the hollow elongate heat transfer elements each including at least three elongate substantially parallel and juxtaposed portions.

28. The catheter of claim 21, the at least one input lumen including at least one input port and the at least one output lumen including at least one output port, the elongate elements being coupled at the first end to the at least one input port and coupled at the second end to the at least one output port, each hollow heat transfer element having a substantially uniform cross section between the input port and the output port.

29. A catheter adapted to exchange heat with a body fluid flowing through a body conduit, the catheter comprising
a shaft including an outer wall and at least one input lumen and at least one output lumen, each lumen extending longitudinally of the shaft within the outer wall;
at least one elongate element having a heat transfer lumen extending longitudinally therethrough with first and second ends, each heat transfer lumen being in fluid communication with one of the at least one input lumen at the first end and in fluid communication with one of the at least one output lumen at the second end, each of the at least one elongate element being folded between the first and second ends into a plurality of elongate and juxtaposed portions extending longitudinally of the shaft.

30. The catheter of claim 29, there being a plurality of the elongate elements.

31. The catheter of claim 29, there being two elongate elements.

32. The catheter of claim 29, there being a plurality of the at least one input lumen, each heat transfer lumen being in fluid communication with one of the input lumens at the first end, respectively.

33. The catheter of claim 32, there being a plurality of the at least one output lumen, each heat transfer lumen being in fluid communication with one of the output lumens at the second end, respectively.

34. The catheter of claim 29, the elongate substantially parallel and juxtaposed portions of each elongate element lying in a plane extending radially from the shaft.

35. The catheter of claim 34, the elongate elements being equiangularly spaced about the shaft.

36. The catheter of claim 34, the elongate elements each being formed as a helix about the shaft.

37. The catheter of claim 29, each elongate substantially parallel portion being bonded to the adjacent elongate substantially parallel portion at the ends thereof.

38. The catheter of claim 29, each elongate substantially parallel portion being thin walled and collapsible under fluid pressure within the body with the heat transfer lumens at atmospheric pressure.

39. The catheter of claim 29, the at least one elongate element including at least three first elongate substantially parallel and juxtaposed portions.

40. The catheter of claim 29, the shaft further including an infusion lumen with an opening at the end of the shaft most adjacent the at least one elongate element.

41. The catheter of claim 29, the at least one input lumen including at least one input port and the at least one output lumen including at least one output port, the at least one elongate element being coupled at the first end to the at least one input port and coupled at the second end to the at least one output port, each heat transfer lumen having a substantially uniform cross section between the input port and the output port.

42. A catheter adapted to exchange heat with a body fluid flowing through a body conduit, the catheter comprising
a shaft including an outer wall and at least one input lumen and at least one output lumen, each lumen extending longitudinally of the shaft within the outer wall;
hollow elongate heat transfer elements, each hollow elongate heat transfer element having first and second ends, being in fluid communication with one of the input lumens, respectively, at the first end, being in fluid communication with one of the output lumens, respectively, at the second end, and being folded between the first and second ends into elongate substantially parallel and juxtaposed portions extending 10 longitudinally of the shaft.

43. The catheter of claim 42, there being two hollow elongate elements.

44. The catheter of claim 42, there being a plurality of the input lumens and a plurality of the output lumens, each hollow element being in fluid communication with one of the input lumens, respectively, and being in fluid communication with one of the output lumens, respectively.

45. The catheter of claim 42, the elongate substantially parallel and juxtaposed portions of each hollow elongate element lying in a plane extending radially from the shaft.

46. The catheter of claim 45, the hollow elongate elements being equiangularly spaced about the shaft.

47. The catheter of claim 45, the hollow elongate elements each being formed as a helix about the shaft.

48. The catheter of claim 42, each elongate substantially parallel portion being bonded to the adjacent elongate substantially parallel portion at the ends thereof.

49. The catheter of claim 42, each elongate substantially parallel portion being thin walled and collapsible under fluid pressure within the body with the heat transfer lumens at atmospheric pressure.

50. The catheter of claim 42, the hollow elongate heat transfer elements each including at least three elongate substantially parallel and juxtaposed portions.

51. The catheter of claim 42, the at least one input lumen including at least one input port and the at least one output lumen including at least one output port, the elongate elements being coupled at the first end to the at least one input port and coupled at the second end to the at least one output port, each hollow heat transfer element having a substantially uniform cross section between the input port and the output port.

52. A catheter adapted to exchange heat with a body fluid flowing through a body conduit, the catheter comprising
a shaft including an outer wall, input lumens and output lumens, each lumen extending longitudinally of the shaft within the outer wall;
hollow elongate heat transfer elements, each hollow elongate heat transfer element having first and second ends, being in fluid communication with one of the input lumens, respectively, at the first end, being in fluid communication with one of the output lumens, respectively, at the second end and having at least one length folded between the first and second ends into at least three elongate substantially parallel and juxtaposed portions, each length extending longitudinally of the shaft and lying in a plane extending radially from the shaft, the planes being equiangularly spaced about the shaft.

53. The catheter of claim 52, the hollow elongate elements each being formed as a helix about the shaft.

54. The catheter of claim 52, each elongate substantially parallel portion being bonded to the adjacent elongate substantially parallel portion at the ends thereof.

55. The catheter of claim 52, each elongate substantially parallel portion being thin walled and collapsible under fluid pressure within the body with the heat transfer lumens at atmospheric pressure.

56. The catheter of claim 52, the at least one input lumen including at least one input port and the at least one output lumen including at least one output port, the elongate elements being coupled at the first end to the at least one input port and coupled at the second end to the at least one output port, each hollow heat transfer element having a substantially uniform cross section between the input port and the output port.

57. Employment of a catheter for heat transfer within the body, comprising
preparing a catheter including at least one input lumen, at least one output lumen and at least one elongate element having a heat transfer lumen extending longitudinally therethrough with first and second ends, each heat transfer lumen being in fluid communication with one of the at least one input lumen at the first end and in fluid communication with one of the at least one output lumen at the second end, a first length of each of the at least one elongate element between the first and second ends being folded into first elongate substantially parallel and juxtaposed portions;
inserting the catheter into a body conduit;
directing liquid through the at least one input lumen, the at least one elongate element and the at least one output lumen in seriatim;
raising the pressure of the liquid to expand fully the at least one elongate element.

58. The employment of a catheter of claim 57, raising the pressure of the liquid including raising the pressure of the liquid to achieve turbulent flow in the at least one elongate element.

59. The employment of a catheter of claim 57, inserting the catheter including inserting the catheter into the neck of the body.

60. The employment of a catheter of claim 57, inserting the catheter including inserting the catheter into the chest of the body.

61. The employment of a catheter of claim 57, inserting the catheter including inserting the catheter into the groin of the body.

62. The employment of a catheter of claim 57 further comprising
cooling the body coincident with cardiac surgery.

63. The employment of a catheter of claim 57 further comprising
cooling the body for fever control.

64. The employment of a catheter of claim 57, preparing a catheter including a sheath thereover, the employment of a catheter further comprising
removing the sheath after inserting the catheter.

65. The employment of a catheter of claim 57, preparing a catheter including a wire guide therethrough, the employment of a catheter further comprising
withdrawing the wire guide after inserting the catheter. 23.
